(19) United States
(12) Patent Application Publication
(10) Pub. No.: US 2026/0034116 A1
(43) Pub. Date: Feb. 5, 2026

(54) CANCER COMBINATION THERAPY INCLUDING A BCL-2 INHIBITOR

(71) Applicants: RYVU THERAPEUTICS S.A., Kraków (PL); BERLIN-CHEMIE AG, Berlin (DE)

(72) Inventors: Monica BINASCHI, Pomezia (IT); Daniela BELLAROSA, Pomezia (IT); Giuseppe MERLINO, Pomezia (IT); Simone BALDINI, Firenze (IT); Tomasz RZYMSKI, Kraków (PL)

(21) Appl. No.: 18/998,815

(22) PCT Filed: Jul. 28, 2023
(86) PCT No.: PCT/EP2023/070946

§ 371 Date: Jan. 27, 2025

(30) Foreign Application Priority Data
Jul. 29, 2022 (EP) 22460034.6

(51) Int. Cl. A61K 31/454; A61K 31/635; A61K 31/706; A61K 31/7068; A61P 35/02

(52) U.S. Cl. CPC A61K 31/454; A61K 31/635; A61K 31/706; A61K 31/7068; A61P 35/02

(57) ABSTRACT

The present invention is inter alia concerned with a combination of (i) a BCL-2 inhibitor and (ii) SEL24/MEN1703 for use as medicament, preferably for use in the treatment of a patient suffering from cancer. The present invention is also concerned with a kit of dosage forms comprising (i) a dosage form comprising a BCL-2 inhibitor and (ii) a dosage form comprising SEL24/MEN1703; as well as dosage form comprising (i) a BCL-2 inhibitor and (ii) SEL24/MEN1703.

### FIELD OF THE INVENTION

The present invention is in the field of cancer therapy. More specifically, the present invention is directed in one aspect to a combination of (i) a BCL-2 inhibitor and (ii) SEL24/MEN1703 for use as medicament. In another aspect, the present invention is concerned with a combination of (i) a BCL-2 inhibitor and (ii) SEL24/MEN1703 for use in the treatment of a patient suffering from cancer. In yet another aspect, the present invention is directed to a kit of dosage forms comprising (i) a dosage form comprising a BCL-2 inhibitor and (ii) a dosage form comprising SEL24/MEN1703. In yet another aspect, the present invention is concerned with a dosage form comprising (i) a BCL-2 inhibitor and (ii) SEL24/MEN1703.

### BACKGROUND OF THE INVENTION

The protein B-cell lymphoma 2 (BCL-2) was first identified in B-cell lymphoma and it was subsequently found that BCL-2 is also upregulated in other hematologic malignancies including acute myeloid leukemia (AML). BCL-2 inhibits pro-apoptotic proteins and thus prevents apoptosis of the AML-cells. Venetoclax was developed as BCL-2 inhibitor with the aim of promoting cell death by apoptosis in that the inhibition of pro-apoptotic proteins is blocked (because BCL-2 is inhibited by Venetoclax). Venetoclax was shown to be effective in treating chronic lymphocytic leukemia (CLL) and was also tested as single agent in AML treatment, where it was found to be safe with a modest antileukemic activity. Because of this rather modest activity when used as monotherapy, studies were initiated of combining Venetoclax with other active agents (see Samra et al. 2020).

Venetoclax combinations that were tested inter alia relate to Venetoclax-hypomethylating agent combinations, a Venetoclax-cytarabine combination (where cytarabine is administered at a low dose), Venetoclax-intensive chemotherapy combinations (e.g. cytarabine and anthracycline), Venetoclax-IDH inhibitor combinations, Venetoclax-FLT3 inhibitor combinations, and Venetoclax-p53-restoring compound combinations (see Samra et al. 2020). When it comes to Venetoclax-hypomethylating agent combinations and the combination of Venetoclax-cytarabine (low dose cytarabine), Venetoclax-products are available today for a specific patient population suffering from AML, namely the products Venclyxto® in Europe and VENCLEXTA® in the US.

Unfortunately, as Samra et al. 2020 put it, despite impressive response rates and improved survival with Venetoclax-combinations, a third of patients seem to be refractory, wherein the most recognized mechanism of resistance to Venetoclax is the upregulation of BCL-2 family antiapoptotic proteins such as BCL-XL and MCL-1 leading to leukemic cell survival. The resistance to Venetoclax and hypomethylating agents in AML is also mentioned and reviewed in Saliba et al. 2021, where it is stated that despite the success of the combination of Venetoclax with the hypomethylating agents (HMA) decitabine or azacitidine in inducing remission in older, previously untreated patients with AML, resistance—primary or secondary—still constitutes a significant roadblock in the quest to prolong the duration of response. Patients unresponsive to Venetoclax plus azacitidine are also discussed in Zhang et al., 2022, where the FLT3 inhibitor gilteritinib was then administered in one patient together with Venetoclax and azacitidine (induction therapy, wherein the maintenance therapy was Venetoclax and gilteritinib), and in the other patient with Venetoclax (induction therapy, wherein the maintenance therapy was Gilteritinib).

Although there has been progress in single case studies, e.g. the two cases in Zhang et al., 2022 discussed above, there is still the general need to overcome resistance to Venetoclax and Venetoclax-combination treatments. This not only applies to Venetoclax but also the BCL-2 inhibitors in general.

### OBJECTS AND SUMMARY OF THE INVENTION

The inventors of the present invention have surprisingly found that the BCL-2 inhibitor-combinations, in particular Venetoclax-combinations, of the present invention are superior over a BCL-2 inhibitor-monotherapy, in particular Venetoclax-monotherapy, and/or existing BCL-2 inhibitor-combinations, in particular Venetoclax-combinations, and therefore provide a very promising novel treatment option for cancer patients.

In a first aspect, the present invention is directed to a combination of (i) a BCL-2 inhibitor and (ii) SEL24/MEN1703 for use as medicament.

In an embodiment, SEL24/MEN1703 is administered at a daily dose of about 50 mg to about 150 mg. It is preferred that SEL24/MEN1703 is administered at a daily dose of about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg. It is most preferred that SEL24/MEN1703 is administered at a daily dose of about 100 mg, about 105 mg, about 110 mg, about 115 mg, about 120 mg or about 125 mg. It is preferred that SEL24/MEN1703 is administered once a day. It is further preferred that SEL24/MEN 1703 is administered orally.

In an embodiment, the BCL-2 inhibitor is selected from the group consisting of Venetoclax, S65487, APG-2575, LOXO-338, ZN-d5, BGB-11417, AZD4320 and AZD0466.

In a preferred embodiment, the BCL-2 inhibitor is S65487, APG-2575, or Venetoclax.

In a more preferred embodiment, the BCL-2 inhibitor is Venetoclax.

### S65487 as the BCL-2 Inhibitor

In an embodiment, S65487 is administered at a therapeutically effective dose. It is preferred that S65487 is administered once a day. It is further preferred that S65487 is administered intravenously.

### APG-2575 as the BCL-2 Inhibitor

In an embodiment, APG-2575 is administered at a therapeutically effective dose, which can be a daily dose of about 100 mg, or about 200 mg, or about 300 mg, or about 400 mg, or about 500 mg, or about 600 mg, or about 700 mg, or about 800 mg. It is preferred that APG-2575 is administered once a day. It is further preferred that APG-2575 is administered orally.

### LOXO-338 as the BCL-2 Inhibitor

In an embodiment, LOXO-338 is administered at a therapeutically effective dose. It is preferred that LOXO-338 is administered once a day. It is further preferred that LOXO-338 is administered orally.

### ZN-d5 as the BCL-2 Inhibitor

In an embodiment, ZN-d5 is administered at a therapeutically effective dose. It is preferred that ZN-d5 is administered once a day. It is further preferred that ZN-d5 is administered orally.

### BGB-11417 as the BCL-2 Inhibitor

In an embodiment, BGB-11417 is administered at a therapeutically effective dose. It is preferred that BGB-11417 is administered once a day. It is further preferred that BGB-11417 is administered orally.

### AZD0466 as the BCL-2 Inhibitor

In an embodiment, AZD0466 is administered at a therapeutically effective dose. It is preferred that AZD0466 is administered once a day. It is further preferred that AZD0466 is administered intravenously.

### AZD4320 as the BCL-2 Inhibitor

In an embodiment, AZD4320 is administered at a therapeutically effective dose. It is preferred that AZD4320 is administered once a day. It is further preferred that AZD4320 is administered intravenously.

### Venetoclax as the BCL-2 Inhibitor

In an embodiment, Venetoclax is administered at a daily dose of about 50 mg to about 600 mg. It is preferred that Venetoclax is administered at a daily dose of about 50 mg, about 100 mg, about 200 mg, about 300 mg, or about 400 mg, or about 500 mg, or about 600 mg. It is most preferred that Venetoclax is administered either at a daily dose of about 350 mg, about 380 mg, or about 400 mg; or at a daily dose of about 550 mg, about 580 mg, or about 600 mg. It is preferred that Venetoclax is administered once a day. Venetoclax may be administered on the first day of a treatment cycle at a daily dose of about 100 mg, followed by a daily dose of about 200 mg on the second day, and followed by a daily dose of about 400 mg on the third day and beyond, or, alternatively to the beyond, followed by a daily dose of about 600 mg on the fourth day and then also with about 600 mg beyond the fourth day. It is further preferred that Venetoclax is administered orally.

In an embodiment, in particular if the BCL-2 inhibitor is Venetoclax, the combination for use of the first aspect further comprises (iii) a hypomethylating agent or cytarabine. Preferably, the hypomethylating agent is selected from the group consisting of azacitidine, decitabine, CC-486 and ASTX727. More preferably, the hypomethylating agent is azacitidine or decitabine, most preferably azacitidine.

In an embodiment, in particular if the BCL-2 inhibitor is Venetoclax, the hypomethylating agent is administered at a daily dose of about 10 mg/m² to about 100 mg/m² (the unit mg/m² as used herein refers to the mg/m² of body surface area [BSA]). It is preferred that the hypomethylating agent is administered at a daily dose of about 10 mg/m², about 20 mg/m², about 30 mg/m², about 40 mg/m², about 50 mg/m², about 60 mg/m², about 70 mg/m², about 80 mg/m², about 90 mg/m², or about 100 mg/m². If the hypomethylating agent is azacitidine, azacitidine is preferably administered at a daily dose of about 60 mg/m², about 65 mg/m², about 70 mg/m², or about 75 mg/m². If the hypomethylating agent is decitabine, decitabine is preferably administered at a daily dose of about 10 mg/m², about 15 mg/m², or about 20 mg/m². It is preferred that the hypomethylating agent is administered once a day. It is further preferred that the hypomethylating agent is administered intravenously, subcutaneously or orally, wherein azacitidine is preferably administered intravenously or subcutaneously, whereas decitabine is preferably administered intravenously.

In an embodiment, in particular if the BCL-2 inhibitor is Venetoclax, the hypomethylating agent is administered for (a) seven consecutive days on days 1 to 7 of a 28-day cycle, or (b) five consecutive days on days 1 to 5 of a 28-day cycle, wherein the hypomethylating agent in (a) is preferably azacitidine and the hypomethylating agent in (b) is preferably decitabine.

In an embodiment, in particular if the BCL-2 inhibitor is Venetoclax, the cytarabine is administered at a daily dose of about 5 mg/m², about 10 mg/m², about 15 mg/m², or about 20 mg/m². It is preferred that the cytarabine is administered at a daily dose of about 20 mg/m². It is further preferred that the cytarabine is administered once a day. It is further preferred that the cytarabine is administered subcutaneously. In an embodiment, the cytarabine is administered for 10 consecutive days on days 1 to 10 of a 28-day cycle. If cytarabine is used in combination with Venetoclax and SEL24/MEN1703, Venetoclax may be administered from day 4 on (i.e. day 4 and beyond) at a daily dose of about 600 mg.

In an alternative embodiment, the combination for use of the first aspect does not comprise the additional administration of chemotherapy.

In an embodiment relating to the combination for use as medicament, (i) and (ii) (as well as optionally (iii)) are administered as separate dosage forms. In this embodiment, the administration may be concomitantly or sequentially. In yet an alternative embodiment relating to the combination for use as medicament, (i) and (ii) (and optionally (iii)) are administered together in a dosage form.

In a second aspect, the present invention is directed to a combination of (i) a BCL-2 inhibitor and (ii) SEL24/MEN1703 for use in the treatment of a patient suffering from cancer.

In a preferred embodiment, the cancer is a hematological cancer. In an even more preferred embodiment, the cancer is a leukemia. In a most preferred embodiment, the cancer is AML. In one embodiment, in particular if the BCL-2 inhibitor is Venetoclax, the AML may be newly diagnosed AML. In one embodiment, in particular if the BCL-2 inhibitor is Venetoclax, the combination of the invention may be used to treat a patient suffering from AML who is ineligible for intensive chemotherapy, preferably an adult patient with newly diagnosed AML who is ineligible for intensive chemotherapy. In another embodiment, in particular if the BCL-2 inhibitor is Venetoclax, the combination of the invention may be used to treat a patient of 75 years or older. In one embodiment, in particular if the BCL-2 inhibitor is Venetoclax, the combination of the invention may be used to treat patients who have comorbidities that preclude use of intensive induction chemotherapy.

When the patient is suffering from AML, the patient suffering from AML may exhibit a FLT3 mutation that results in overactivation of FLT3 signalling. The mutation in the FLT3 may even result in constitutively active FLT3 signalling (in the meaning that the signaling activity of FLT3 is constitutively active). The FLT3 mutation is caused by at least one base mutation in the FLT3 gene, resulting in the afore-mentioned FLT3 mutation on a protein level that results in overactivation of FLT3 signaling. Such mutations are known in the field for more than 25 years now, with the most common FLT3-ITD (“internal tandem duplication”) mutation reported first in 1996 (see Nakao M et al., 1996). The FLT3 mutation may be a FLT3-ITD mutation, a FLT3-TKD mutation, or a combination of a FLT3-ITD mutation and FLT3-TKD mutation. Additionally or alternatively, the patient suffering from cancer, including a patient suffering from AML, may exhibit at least one IDH1 and/or IDH2 mutation, preferably at least two IDH1 and/or IDH2 mutations. Alternatively, when the patient is suffering from AML, the patient suffering from AML may exhibit a FLT3 wt-status.

In an embodiment, SEL24/MEN1703 is administered at a daily dose of about 50 mg to about 150 mg. It is preferred that SEL24/MEN1703 is administered at a daily dose of about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg. It is most preferred that SEL24/MEN1703 is administered at a daily dose of about 100 mg, about 105 mg, about 110 mg, about 115 mg, about 120 mg or about 125 mg. It is preferred that SEL24/MEN1703 is administered once a day. It is further preferred that SEL24/MEN 1703 is administered orally.

In an embodiment, the BCL-2 inhibitor is selected from the group consisting of Venetoclax, S65487, APG-2575, LOXO-338, ZN-d5, BGB-11417, AZD4320 and AZD0466.

In a preferred embodiment, the BCL-2 inhibitor is S65487, APG-2575, or Venetoclax.

In a more preferred embodiment, the BCL-2 inhibitor is Venetoclax.

### S65487 as the BCL-2 Inhibitor

In an embodiment, S65487 is administered at a therapeutically effective dose. It is preferred that S65487 is administered once a day. It is further preferred that S65487 is administered intravenously.

### APG-2575 as the BCL-2 Inhibitor

In an embodiment, APG-2575 is administered at a therapeutically effective dose, which can be a daily dose of about 100 mg, or about 200 mg, or about 300 mg, or about 400 mg, or about 500 mg, or about 600 mg, or about 700 mg, or about 800 mg. It is preferred that APG-2575 is administered once a day. It is further preferred that APG-2575 is administered orally.

### LOXO-338 as the BCL-2 Inhibitor

In an embodiment, LOXO-338 is administered at a therapeutically effective dose. It is preferred that LOXO-338 is administered once a day. It is further preferred that LOXO-338 is administered orally.

### ZN-d5 as the BCL-2 Inhibitor

In an embodiment, ZN-d5 is administered at a therapeutically effective dose. It is preferred that ZN-d5 is administered once a day. It is further preferred that ZN-d5 is administered orally.

### BGB-11417 as the BCL-2 Inhibitor

In an embodiment, BGB-11417 is administered at a therapeutically effective dose. It is preferred that BGB-11417 is administered once a day. It is further preferred that BGB-11417 is administered orally.

### AZD0466 as the BCL-2 Inhibitor

In an embodiment, AZD0466 is administered at a therapeutically effective dose. It is preferred that AZD0466 is administered once a day. It is further preferred that AZD0466 is administered intravenously.

### AZD4320 as the BCL-2 Inhibitor

In an embodiment, AZD4320 is administered at a therapeutically effective dose. It is preferred that AZD4320 is administered once a day. It is further preferred that AZD4320 is administered intravenously.

### Venetoclax as the BCL-2 Inhibitor

In an embodiment, Venetoclax is administered at a daily dose of about 50 mg to about 600 mg. It is preferred that Venetoclax is administered at a daily dose of about 50 mg, about 100 mg, about 200 mg, about 300 mg, or about 400 mg, or about 500 mg, or about 600 mg. It is most preferred that Venetoclax is administered either at a daily dose of about 350 mg, about 380 mg, or about 400 mg; or at a daily dose of about 550 mg, about 580 mg, or about 600 mg. It is preferred that Venetoclax is administered once a day. Venetoclax may be administered on the first day of a treatment cycle at a daily dose of about 100 mg, followed by a daily dose of about 200 mg on the second day, and followed by a daily dose of about 400 mg on the third day and beyond, or, alternatively to the beyond, followed by a daily dose of about 600 mg on the fourth day and then also with about 600 mg beyond the fourth day. It is further preferred that Venetoclax is administered orally.

In an embodiment, in particular if the BCL-2 inhibitor is Venetoclax, the combination for use of the second aspect further comprises (iii) a hypomethylating agent or cytarabine. Preferably, the hypomethylating agent is selected from the group consisting of azacitidine, decitabine, CC-486 and ASTX727. More preferably, the hypomethylating agent is azacitidine or decitabine, most preferably azacitidine.

In an embodiment, in particular if the BCL-2 inhibitor is Venetoclax, the hypomethylating agent is administered at a daily dose of about 10 mg/m² to about 100 mg/m² (the unit mg/m² as used herein refers to the mg/m² of body surface area [BSA]). It is preferred that the hypomethylating agent is administered at a daily dose of about 10 mg/m², about 20 mg/m², about 30 mg/m², about 40 mg/m², about 50 mg/m², about 60 mg/m², about 70 mg/m², about 80 mg/m², about 90 mg/m², or about 100 mg/m². If the hypomethylating agent is azacitidine, azacitidine is preferably administered at a daily dose of about 60 mg/m², about 65 mg/m², about 70 mg/m², or about 75 mg/m². If the hypomethylating agent is decitabine, decitabine is preferably administered at a daily dose of about 10 mg/m², about 15 mg/m², or about 20 mg/m². It is preferred that the hypomethylating agent is administered once a day. It is further preferred that the hypomethylating agent is administered intravenously, subcutaneously or orally, wherein azacitidine is preferably administered intravenously or subcutaneously, whereas decitabine is preferably administered intravenously.

In an embodiment, in particular if the BCL-2 inhibitor is Venetoclax, the hypomethylating agent is administered for (a) seven consecutive days on days 1 to 7 of a 28-day cycle, or (b) five consecutive days on days 1 to 5 of a 28-day cycle, wherein the hypomethylating agent in (a) is preferably azacitidine and the hypomethylating agent in (b) is preferably decitabine.

In an embodiment, in particular if the BCL-2 inhibitor is Venetoclax, the cytarabine is administered at a daily dose of about 5 mg/m², about 10 mg/m², about 15 mg/m², or about 20 mg/m². It is preferred that the cytarabine is administered at a daily dose of about 20 mg/m². It is further preferred that the cytarabine is administered once a day. It is further preferred that the cytarabine is administered subcutaneously. In an embodiment, the cytarabine is administered for 10 consecutive days on days 1 to 10 of a 28-day cycle. If cytarabine is used in combination with Venetoclax and SEL24/MEN1703, Venetoclax may be administered from day 4 on (i.e. day 4 and beyond) at a daily dose of about 600 mg.

In an embodiment, the combination for use of the second aspect does not comprise the additional administration of chemotherapy.

In an embodiment relating to the combination for use as medicament, (i) and (ii) (as well as optionally (iii)) are administered as separate dosage forms. In this embodiment, the administration may be concomitantly or sequentially. In yet an alternative embodiment relating to the combination for use as medicament, (i) and (ii) (and optionally (iii)) are administered together in a dosage form.

In a preferred embodiment of the second aspect, the combination comprises (i) Venetoclax as the BCL-2 inhibitor and (ii) SEL24/MEN1703 as the sole active agents; the cancer is AML; Venetoclax is administered (after an initial titration starting from about 100 mg on day 1 to about 200 mg on day 2) at a daily dose of about 400 mg (starting from day 3), preferably orally; SEL24/MEN1703 is administered at a daily dose of about 80 mg to about 120 mg, preferably orally. It can be preferred in this embodiment that the patient suffering from AML is an adult patient with newly diagnosed AML, who might be ineligible for intensive chemotherapy.

In yet another preferred embodiment, the combination comprises (i) Venetoclax as the BCL-2 inhibitor, (ii) SEL24/MEN1703, and (iii) a hypomethylating agent, preferably azacitidine or decitabine; the cancer is AML; Venetoclax is administered (after an initial titration starting from about 100 mg on day 1 to about 200 mg on day 2) at a daily dose of about 400 mg (starting from day 3), preferably orally; SEL24/MEN1703 is administered at a daily dose of about 80 mg to about 120 mg, preferably orally; the hypomethylating agent is administered at a daily dose of about 20 mg/m² to about 75 mg/m² (with about 75 mg/m² being preferred for azacitidine and about 20 mg/m² being preferred for decitabine), preferably intravenously. It can be preferred in this embodiment that the patient suffering from AML is an adult patient with newly diagnosed AML, who might be ineligible for intensive chemotherapy.

In yet another preferred embodiment, the combination comprises (i) Venetoclax as the BCL-2inhibitor, (ii) SEL24/MEN1703, and (iii) cytarabine; the cancer is AML; Venetoclax is administered (after an initial titration starting from about 100 mg on day 1 to about 200 mg on day 2 and about 400 mg on day 3) at a daily dose of about 600 mg (starting from day 4), preferably orally; SEL24/MEN1703 is administered at a daily dose of about 80 mg to about 120 mg, preferably orally; cytarabine is administered at a daily dose of about 20 mg/m2, preferably subcutaneously. It can be preferred in this embodiment that the AML is newly diagnosed AML, and the patient is 75 years or older. In one embodiment, the combination may be used to treat a patient (optionally with newly diagnosed AML) who have comorbidities that preclude use of intensive induction chemotherapy.

In a third aspect, the present invention is directed to a kit of dosage forms comprising (i) a dosage form comprising a BCL-2 inhibitor and (ii) a dosage form comprising SEL24/MEN1703.

In an embodiment, the dosage form comprising a BCL-2 inhibitor comprises a BCL-2 inhibitor selected from the group consisting of Venetoclax, S65487, APG-2575, LOXO-338, ZN-d5, BGB-11417, AZD4320 and AZD0466.

In a preferred embodiment, the dosage form comprising a BCL-2 inhibitor comprises a BCL-2 inhibitor selected from the group consisting of S65487, APG-2575 and Venetoclax.

In a more preferred embodiment, the dosage form comprising a BCL-2 inhibitor comprises Venetoclax as the BCL2-inhibitor.

In an embodiment, the dosage form comprising a BCL-2 inhibitor comprises Venetoclax and comprises Venetoclax in an amount of about 50 mg to about 600 mg, preferably in an amount of about 50 mg, about 100 mg, about 200 mg, about 300 mg, or about 400 mg, more preferably in an amount of about 350 mg, about 380 mg, or about 400 mg; or in an amount of about 550 mg, about 580 mg, or about 600 mg. It is preferred that the dosage form comprising Venetoclax is a once-a-day dosage form. It is further preferred that the dosage form comprising Venetoclax is an oral dosage form.

In an embodiment, the dosage form comprising SEL24/MEN1703 comprises SEL24/MEN1703 in an amount of about 50 mg to about 150 mg, preferably in an amount of about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg, most preferably in an amount of about 100 mg, about 105 mg, about 110 mg, about 115 mg, about 120 mg or about 125 mg. It is preferred that the dosage form comprising SEL24/MEN1703 is a once-a-day dosage form. It is further preferred that the dosage form comprising SEL24/MEN1703 is an oral dosage form.

Each dosage form typically contains at least one pharmaceutically acceptable excipient as defined in section 2 of the detailed description below.

In particular if Venetoclax is comprised in the kit as BCL-2 inhibitor, the kit may further comprise in an embodiment a dosage form comprising a hypomethylating agent, preferably azacitidine or decitabine, or a dosage form comprising cytarabine, wherein the respective dosage forms comprise the corresponding active in an amount required to achieve the administration levels (mg/m² body surface area [BSA], as outlined above) as outlined above in the second aspect, together with instructions on how to prepare the corresponding intravenous or subcutaneous administration dosage forms. Preferably, the kit further comprises a dosage form comprising azacitidine.

In an embodiment, the kit of the third aspect further comprises a leaflet setting out the instructions how to use and administer the dosage forms.

In a fourth aspect, the present invention is directed to a dosage form comprising (i) a BCL-2 inhibitor and (ii) SEL24/MEN1703.

In an embodiment, the dosage form comprises a BCL-2 inhibitor selected from the group consisting of Venetoclax, S65487, APG-2575, LOXO-338, ZN-d5, BGB-11417, AZD4320 and AZD0466.

In a preferred embodiment, the dosage form comprises a BCL-2 inhibitor selected from the group consisting of S65487, APG-2575 and Venetoclax.

In a more preferred embodiment, the dosage form comprises Venetoclax as the BCL2-inhibitor.

In an embodiment, the dosage form comprises Venetoclax as the BCL-2 inhibitor and comprises Venetoclax in an amount of about 50 mg to about 600 mg, preferably in an amount of about 50 mg, about 100 mg, about 200 mg, about 300 mg, or about 400 mg, more preferably in an amount of about 350 mg, about 380 mg, or about 400 mg; or in an amount of about 550 mg, about 580 mg, or about 600 mg. It is preferred that the dosage form comprising Venetoclax is a once-a-day dosage form. It is further preferred that the dosage form comprising Venetoclax is an oral dosage form.

In an embodiment, the dosage form comprises SEL24/MEN1703 in an amount of about 50 mg to about 150 mg, preferably in an amount of about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg, most preferably in an amount of about 100 mg, about 105 mg, about 110 mg, about 115 mg, about 120 mg or about 125 mg.

In particular if Venetoclax is comprised in the dosage form as BCL-2 inhibitor, the dosage form further comprises in an embodiment a hypomethylating agent, preferably CC-486 or ASTX727 due to their oral administration.

In an embodiment, the dosage form is a once-a-day dosage form. It is further preferred that the dosage form is an oral dosage form.

The dosage form typically contains at least one pharmaceutically acceptable excipient as defined in section 2 of the detailed description below.

In an embodiment, the dosage form of the fourth aspect comes with a leaflet setting out the instructions how to use and administer the dosage form.

In a fifth aspect, the present invention is directed to a method of treating cancer in a patient in need thereof, said method comprising administering to the patient an effective amount of (i) a BCL-2 inhibitor and an effective amount of (ii) SEL24/MEN1703.

All embodiments outlined above for the second aspect equally apply for the fifth aspect.

### DESCRIPTION OF THE FIGURES

FIG. 1: Statistical analysis of cytotoxicity data at IC75 concentrations in KG1 cells. When the triple combination Men+Aza+Ven (mav) was compared with Men+Ven, Ven+Aza, Men+Aza and the single agent treatments, a significant difference was found for the Men+Aza+Ven treatment vs. Men+Ven and vs. single treatments (Student's t test, p<0.01**, p<0.1*). The Ven+Men combination showed a significant difference vs. the Ven single treatment.

FIG. 2: Statistical analysis of cytotoxicity data at IC75 concentrations in MV4-11 cells. When the triple combination Men+Aza+Ven (mav) was compared with Men+Ven, Ven+Aza, Men+Aza and the single agent treatments, a significant difference was found for the Men+Aza+Ven treatment vs. Men alone and for the Men+Ven combination treatments vs. Ven as single agent treatment (Student's t test, p<0.01**, p<0.1*).

FIG. 3: In vivo studies in an AML MOLM-16 cell line xenograft. Antitumor activity of MEN1703, 5-Azacytidine and Venetoclax as single agents (left graph) and as double and triple drugs combinations (right graph) in the MOLM-16 xenograft tumor model. Tumor cells were injected s.c. into SCID mice at day 0 and drug dosing started on day 29. The first arrows under the x-axis represent the 5-Azacytidine administration, the second arrows under the x-axis represent the MEN1703 administration, and the third arrows under the x-axis represent the Venetoclax administration. (A) shows the tumor volume for single treatments (left graph) as well as the combination treatments (right graph) over all days of the respective administrations, whereas (B) shows the tumor volume at day 41.

FIG. 4: In vivo studies in an AML MV4-11 cell line xenograft. Antitumor activity of MEN 1703, 5-Azacytidine and Venetoclax as single agents (left graph) and as double and triple drugs combi-nations (right graph) in the MV4-11 xenograft tumor model. Tumor cells were injected s.c. into SCID mice at day 0 and drug dosing started on day 21. The first arrows under the x-axis represent the 5-Azacytidine administration, the second arrows under the x-axis represent the MEN1703 administration, and the third arrows under the x-axis represent the Venetoclax administration. (A) shows the tumor volume for single treatments (left graph) as well as the combination treatments (right graph) over all days of the respective administrations, whereas (B) shows the tumor volume at day 45.

FIG. 5: Engraftment of human hematopoietic cells (CD45 positive) in the peripheral blood of NSG mice monitored at the indicated time points.

FIG. 6: The histogram represents the percentage of human CD45+ AML cells engraftment in mice, 110 days post transplantation. Results are expressed as mean±SD (**p<0.01; ***p<0.001; ****p<0.0001).

FIG. 7: Kaplan-Meier curves comparing overall survival of PDX AML mice models and statistical results of the treatment groups comparison.

FIG. 8: Statistical analysis of cytotoxicity data at IC50 concentrations in (A) MOLM-13 cells and (B) MV4-11 cells (“MEN 1703”=MEN, “MEN1703+S65487”=“MEN+S65487”). The MEN1703+S65487 combination induced a cytotoxicity significantly different from the MEN1703-induced cytotoxicity and the S65487-induced cytotoxicity (Tukey's multiple comparison one-way ANOVA test; p<0.05*, p<0.01**, p<0.001***).

FIG. 9: Statistical analysis of cytotoxicity data at IC50 concentrations in (A) MOLM-13 cells and (B) MV4-11 cells (“MEN1703”=MEN, “MEN1703+AZD4320”=“MEN+AZD4320”). The MEN1703+AZD4320 combination induced a cytotoxicity significantly different from the MEN1703-induced cytotoxicity and the AZD4320-induced cytotoxicity (Tukey's multiple comparison one-way ANOVA test; p<0.05*).

### DETAILED DESCRIPTION OF THE INVENTION

Before the present invention is described in more detail, the following definitions are introduced.

### 1. Definitions

As used in the specification and the claims, the singular forms of “a” and “an” also include the corresponding plurals unless the context clearly dictates otherwise.

The term “about” in the context of the present invention denotes an interval of accuracy that a person skilled in the art will understand to still ensure the technical effect of the feature in question. The term typically indicates a deviation from the indicated numerical value of ±10% and preferably ±5%.

It needs to be understood that the term “comprising” is not limiting. For the purposes of the present invention, the term “consisting of” is considered to be a preferred embodiment of the term “comprising”. If hereinafter a group is defined to comprise at least a certain number of embodiments, this is also meant to encompass a group which preferably consists of these embodiments only.

The term “a combination” or “in combination with” as used herein is not intended to imply that the therapy or the active agents (i) and (ii) (as well as optionally (iii) must be administered at the same time and/or formulated for delivery together, although such therapy and formulations are within the scope of the present invention. The active agents in the combination can be administered concurrently with, prior to, or subsequent to, each other, and even one or more other additional therapies or active agents. The active agents or therapeutic protocol can be administered in any order. In general, each active agent will be administered at a dose and/or on a time schedule determined for that active agent. Further, in general, it is expected that active agents used in combination are used at doses that do not exceed the doses at which they are used individually. In some embodiments, the doses used in the combination will be lower than the doses used individually. In some embodiments, one of the two active agents is administered at a therapeutic or lower-than therapeutic dose, e.g. Venetoclax is administered at a lower-than therapeutic dose or SEL24/MEN1703 is administered at a lower-than therapeutic dose (wherein the “lower-than therapeutic dose” is derived from a comparison to the therapeutic dose as single active agent in a monotherapy). A lower-than therapeutic dose can e.g. be 10-20%, 20-30%, 30-40%, 40-50%, 50-60%, 60-70%, 70-80%, or 80-90% lower than the corresponding monotherapy.

The term “Venetoclax” as used herein means the BCL-2 inhibitor Venetoclax, marketed under the tradename Venclyxto® (EMA) and Venclexta® (FDA), which are both authorized inter alia for AML-treatment. Further details about Venetoclax can inter alia be found in the product leaflets or the regulatory dossiers.

The term “SEL24/MEN1703” (alternatively referred to herein as “MEN”, “Men”, “men” or “Men1703”) as used herein means the compound 5,6-dibromo-4-nitro-2-(piperidin-4-yl)-1-(pro-pan-2-yl)-1H-1,3-benzodiazole, in the form of the free base or a pharmaceutically acceptable salt thereof (such as the HCl-salt). The free base form has the CAS-number 1616359-00-2, whereas the HCl-salt form has the CAS-number 2769008-22-0. The compound is a dual pan-PIM/FLT3 inhibitor, which has inter alia been shown to inhibit the growth of a broad panel of AML cell lines in xenograft models. The rationale for the development of this dual inhibitor was that PIM kinases are deemed to be major drivers of the resistance to FLT3-inhibitors. SEL24/MEN 1703 is characterized in more detail e.g. in Czardybon et al., 2018. WO 2014/096388 discloses SEL24/MEN1703 as Compound 26A therein and characterizes SEL24/MEN1703 as dual pan-PIM/FLT3 inhibitor, see Table 1A of WO 2014/096388, which is also the characterization in Czardybon et al., 2018. WO 2014/096388 fails to disclose a combination of SEL24/MEN1703 with a BCL-2 inhibitor. Furthermore, it is evident from the data of WO 2014/096388 that the different compounds synthesized and tested therein do not necessarily share the same mechanism of action (e.g. when comparing Compound 26A to other compounds, e.g. Compound 1A structurally differing in the ring substituent and the alkyl substituent from Compound 26A, with respect to their activities towards the different PIM-kinases, see Table 1A of WO 2014/096388, from which it can be concluded that SEL24/MEN 1703 is a pan-PIM inhibitor, whereas other compounds, e.g. Compound 1A, are rather specific for certain PIM-kinases).

The term “hypomethylating agent” as used herein means an agent that inhibits DNA methylation, i.e. the modification of DNA nucleotides by the addition of a methyl group. Treatment with a hypomethylating agent is considered to be an epigenetic therapy. The currently available hypomethylating agents including azacitidine (alternatively referred to herein as “5-azacitidine”) and decitabine as disclosed herein block the activity of DNA methyltransferases. Further details about azacitidine and decitabine can inter alia be found in the product leaflets or the regulatory dossiers of Venclyxto® and Venclexta® mentioned above. CC-486 is also a hypomethylating agent, namely oral azacitidine, see Kipp and Wei, 2021. Yet another hypomethylating agent is ASTX727, which is decitabine/cedazuridine, see Kipp and Wei, 2021. CC-486 and ASTX727 are both orally administered hypomethylating agents.

The term “cytarabine” as used herein refers to a chemotherapeutic agent, which is alternatively named “cytosine arabinoside (ara-C)”. Cytarabine combines a cytosine base with an arabinose sugar and interferes with the synthesis of DNA. It does this by its rapid conversion into cytosine arabinoside triphosphate, which damages DNA when the cell cycle holds in the S-phase. Accordingly, rapidly dividing cells are most affected. Further details about cytarabine can inter alia be found in the product leaflet or the regulatory dossiers of Venclexta® mentioned above.

The term “S65487” as used herein refers to the BCL-2 inhibitor S65487, which is alternatively referred to as “VOB560”. It is commercially available e.g. from MedChemExpress and has the CAS-number 1644600-79-2. S65487 is inter alia presently undergoing a clinical study in order to assess safety, tolerability, PK and preliminary clinical activity and to estimate the maximum tolerated doses/recommended phase 2 doses as single agent administered intravenously in adult patients with refractory or relapsed AML, Non-Hodgkin Lymphoma (NHL), Multiple Myeloma (MM) or Chronic Lymphocytic Leukemia (CCL) [ClinicalTrials.gov Identifier: NCT03755154].

The term “APG-2575” as used herein refers to the BCL-2 inhibitor APG-2575, which is alternatively referred to as “Lisaftoclax”. It is commercially available e.g. from MedChemExpress and has the CAS-number 2180923 May 9. APG-2575 is inter alia presently undergoing a clinical study in order to assess the safety, pharmacokinetic of APG-2575 single agent and in combination with homoharringtonine or Azacitidine in patients with relapsed/refractory AML and related myeloid malignancies [ClinicalTrials.gov Identifier: NCT04501120].

The term “LOXO-338” as used herein refers to the BCL-2 inhibitor LOXO-338, which is alternatively referred to as “LY3847429”. LOXO-338 is inter alia presently undergoing a clinical study in order to assess LOXO 338 as monotherapy in patients with advanced hematologic malignancies [ClinicalTrials.gov Identifier: NCT05024045].

The term “ZN-d5” as used herein refers to the BCL-2 inhibitor ZN-d5. Zentalis announced on 30 Apr. 2020 FDA clearance of IND Application for ZN-d5 for the treatment of hematological malignancies.

The term “BGB-11417” as used herein refers to the BCL-2 inhibitor BGB-11417. BGB-11417 is inter alia presently undergoing a clinical study in order to assess BGB-11417 with respect to safety and tolerability, and to define the maximum tolerated dose and recommended phase 2 dose, and to evaluate the safety and tolerability of the ramp-up dosing schedule of the monotherapy [ClinicalTrials.gov Identifier: NCT04277637].

The term “AZD0466” as used herein refers to the BCL-2 inhibitor AZD0466, which is characterized inter alia in Arulananda et al., 2021. AZD0466 is disclosed in a clinical study in order to assess AZD0466 with respect to safety, tolerability, maximum tolerated dose, recommended phase 2 dose and PK in patients with solid tumors, lymphoma and multiple myeloma at low risk for tumor lysis syndrome [ClinicalTrials.gov Identifier: NCT04214093].

The term “AZD4320” as used herein refers to the BCL-2 inhibitor AZD4320, which is characterized inter alia in Balachander et al., 2020. It is commercially available e.g. from MedChemExpress and has the CAS-number 1357576-48-7.

The term “treatment” as used herein refers to clinical intervention in order to cure or ameliorate a disease, prevent recurrence of a disease, alleviate symptoms of a disease, diminish any direct or indirect pathological consequences of a disease, achieve a stabilized (i.e., not worsening) state of disease, prevent metastasis, decrease the rate of disease progression, and/or prolong survival as compared to expected survival if not receiving treatment.

### 2. Pharmaceutical Compositions

A BCL-2 inhibitor, SEL24/MEN1703, the hypomethylating agent and cytarabine are “pharmaceutically active agents” or “active agents” for the purposes of the present invention. As noted above, a BCL-2 inhibitor and SEL247MEN1703 may either be present in separate dosage forms or comprised in a single dosage form.

“Pharmaceutically active agents” as used herein means that the compounds are potent of modulating a response in a patient, i.e. a human or animal being in vivo. The term “pharmaceutically acceptable excipient” as used herein refers to excipients commonly comprised in pharmaceutical dosage forms or compositions, which are known to the skilled person. Such excipients are exemplary listed below. In view of the definition “pharmaceutically active agents” as given above, a pharmaceutically acceptable excipient can be defined as being pharmaceutically inactive.

It is typically preferred that the administration of a BCL-2 inhibitor and SEL24/MEN1703 occurs via separate dosage forms and that a BCL-2 inhibitor is administered in the dosage form and via the administration route that is approved. SEL24/MEN 1703 may be administered in a dosage form as set out in the following or in a dosage form in which it is currently undergoing clinical testing.

A dosage form for use according to the present invention may be formulated for oral, buccal, nasal, rectal, topical, transdermal or parenteral application. Oral application is particularly preferred. Parenteral application includes intravenous, intramuscular or subcutaneous administration. A dosage form of the present invention may also be designated as formulation or pharmaceutical composition.

In general, a pharmaceutical composition according to the present invention can comprise various pharmaceutically acceptable excipients which will be selected depending on which functionality is to be achieved for the composition. A “pharmaceutically acceptable excipient” in the meaning of the present invention can be any substance used for the preparation of pharmaceutical dosage forms, including coating materials, film-forming materials, fillers, disintegrating agents, release-modifying materials, carrier materials, diluents, binding agents and other adjuvants. Typical pharmaceutically acceptable excipients include substances like sucrose, mannitol, sorbitol, starch and starch derivatives, lactose, and lubricating agents such as magnesium stearate, disintegrants and buffering agents.

The term “carrier” denotes pharmaceutically acceptable organic or inorganic carrier substances with which the active ingredient is combined to facilitate the application. Suitable pharmaceutically acceptable carriers include, for instance, water, salt solutions, alcohols, oils, preferably vegetable oils, polyethylene glycols, gelatin, lactose, amylose, magnesium stearate, surfactants, perfume oil, fatty acid monoglycerides and diglycerides, petroethral fatty acid esters, hydroxymethyl-cellulose, polyvinylpyrrolidone and the like. The pharmaceutical compositions can be sterilized and if desired, mixed with auxiliary agents, like lubricants, preservatives, stabilizers, wetting agents, emulsifiers, salts for influencing osmotic pressure, buffers, colorings, flavoring and/or aromatic substances and the like which do not deleteriously react with the active compound.

If liquid dosage forms are considered for the present invention, these can include pharmaceutically acceptable emulsions, solutions, suspensions and syrups containing inert diluents commonly used in the art such as water. These dosage forms may contain e.g. microcrystalline cellulose for imparting bulk, alginic acid or sodium alginate as a suspending agent, methylcellulose as a viscosity enhancer and sweeteners/flavouring agents.

For parenteral application, particularly suitable vehicles consist of solutions, preferably oily or aqueous solutions, as well as suspensions, emulsions, or implants. Pharmaceutical formulations for parenteral administration are particularly preferred and include aqueous solutions in water-soluble form. Additionally, suspensions may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran.

For injectable preparations, sterile injectable aqueous or oleaginous suspensions can for example be formulated according to the known art using suitable dispersing agents, wetting agents and/or suspending agents. A sterile injectable preparation can also be a sterile injectable solution or suspension in a non-toxic parenterally acceptable diluent or solvent. Among the acceptable vehicles and solvents that can be used are water and isotonic sodium chloride solution. Sterile oils are also conventionally used as solvent or suspending medium.

Suppositories for rectal administration of a pharmaceutical composition of the present invention can be prepared by e.g. mixing the compound with a suitable non-irritating excipient such as cocoa butter, synthetic triglycerides and polyethylene glycols which are solid at room temperature but liquid at rectal temperature such that they will melt in the rectum and release the active agent from said suppositories.

For administration by inhalation, the pharmaceutical composition according to the present invention may be conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Oral dosage forms may be liquid or solid and include e.g. tablets, troches, pills, capsules, powders, effervescent formulations, dragees and granules. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. The oral dosage forms may be formulated to ensure an immediate release of the active agent or a sustained release of the active agent.

### 3. Examples

The following Examples are merely illustrative and shall describe the present invention in a further way. These Examples shall not be construed to limit the present invention thereto.

### Example 1: In Vitro Studies in Cell Lines

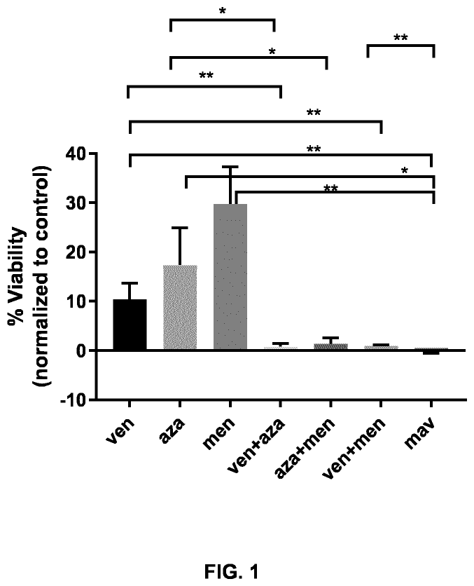

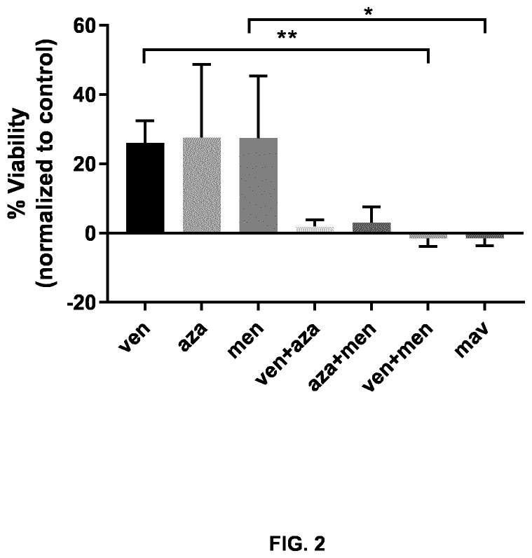

In an in vitro standard cytotoxicity assay, three AML cell lines (KG1 [FLT3 wt], MV4-11 [FLT3 ITD] and MOLM-13 [FLT3 ITD]) were treated with Ven and/or 5-Aza for 48 hours, followed by Men treatment for additional 24 hours. The synergistic effect of the resulting combinations of Men either with Ven or 5-Aza alone (i.e. two actives combined) or with Ven+5-Aza (i.e. three actives combined) was measured according to the Combination Index calculation (Chou TC, 2006). The triple combination of Men+5-Aza+Ven (mav) demonstrated high synergism in most of the tested AML cell lines (see Tables 1 to 3 and FIGS. 1 and 2). In KG1 cells (FLT3 wt), the cytotoxic effect of Men+5-Aza+Ven (mav) treatment was significantly stronger than Men+Ven and single agent treatments (see FIG. 1), wherein the combination index appeared to be lower in the triple combination Men+5-Aza+Ven vs. Men+Ven and Ven+Aza, all applied at clinically relevant concentrations (see Table 1). In MV4-11 cells (FLT3 ITD), a similar trend was observed, wherein the CI of the triple combination was about equal to the CI of Men+Ven and significantly lower than the CI of Ven+Aza (see FIG. 2 and Table 2). In MOLM13 cells (FLT3 ITD), the CI of the triple combination was similar to the CIs of the Men-Ven and the VenAza combinations (see Table 3).

TABLE 1
in vitro Combination Index in KG1 cells

| | Men dose | Ven dose | Aza dose | Combination | Combination | Combination |
|---|---|---|---|---|---|---|
| Men-Ven-Aza | level | level | level | Index (CI) | Index (CI) | Index (CI) |
| dose level | (μM) | (μM) | (μM) | Men + Ven + Aza | Men + Ven | Ven + Aza |
| IC10 | 0.151 | 0.18 | 0.097 | 0.54 | 0.5 | 0.71 |
| IC25 | 0.293 | 0.55 | 0.27 | 0.43 | 0.89 | 0.59 |
| IC50 | 0.569 | 1.65 | 0.76 | 0.13 | 0.29 | 0.66 |
| IC75 | 1.103 | 4.95 | 2.15 | <0.001 | 0.08 | 0.19 |

The combination of Men + Ven + Aza (mav) was strongly synergistic at clinically relevant concentrations (grey rows).

TABLE 2
in vitro Combination Index in MV4-11 cells

| | Men dose | Ven dose | Aza dose | Combination | Combination | Combination |
|---|---|---|---|---|---|---|
| Men-Ven-Aza | level | level | level | Index (CI) | Index (CI) | Index (CI) |
| dose level | (μM) | (μM) | (μM) | Men + Ven + Aza | Men + Ven | Ven + Aza |
| IC10 | 0.006 | 2.76 × 10⁻⁴ | 0.14 | 0.45 | 0.13 | 4.6 |
| IC25 | 0.048 | 8.29 × 10⁻⁴ | 0.3 | 0.31 | 0.33 | 0.6 |
| IC50 | 0.366 | 0.003 | 0.65 | 0.03 | 0.49 | 0.77 |
| IC75 | 2.77 | 0.007 | 1.41 | 0.09 | 0.09 | 0.97 |

The combination of Men + Ven + Aza (mav) was strongly synergistic at clinically relevant concentrations (grey rows).

TABLE 3
in vitro Combination Index in MOLM13 cells

| | Men dose | Ven dose | Aza dose | Combination | Combination | Combination |
|---|---|---|---|---|---|---|
| Men-Ven-Aza | level | level | level | Index (CI) | Index (CI) | Index (CI) |
| dose level | (μM) | (μM) | (μM) | Men + Ven + Aza | Men + Ven | Ven + Aza |
| IC10 | 0.04 | 0.012 | 0.083 | 0.15 | 0.11 | 0.21 |
| IC25 | 0.28 | 0.075 | 0.28 | 0.22 | 0.12 | 0.24 |
| IC50 | 1.73 | 0.45 | 0.96 | 0.43 | 0.19 | 0.46 |
| IC75 | 10.88 | 2.77 | 3.28 | 0.07 | 0.72 | 0.82 |

The CI of the combination of Men + Ven + Aza (mav) was similar to the CI of the Men + Ven and Ven + Aza combinations at a clinically relevant concentration (grey row).

### Example 2: In Vivo Xenograft Studies

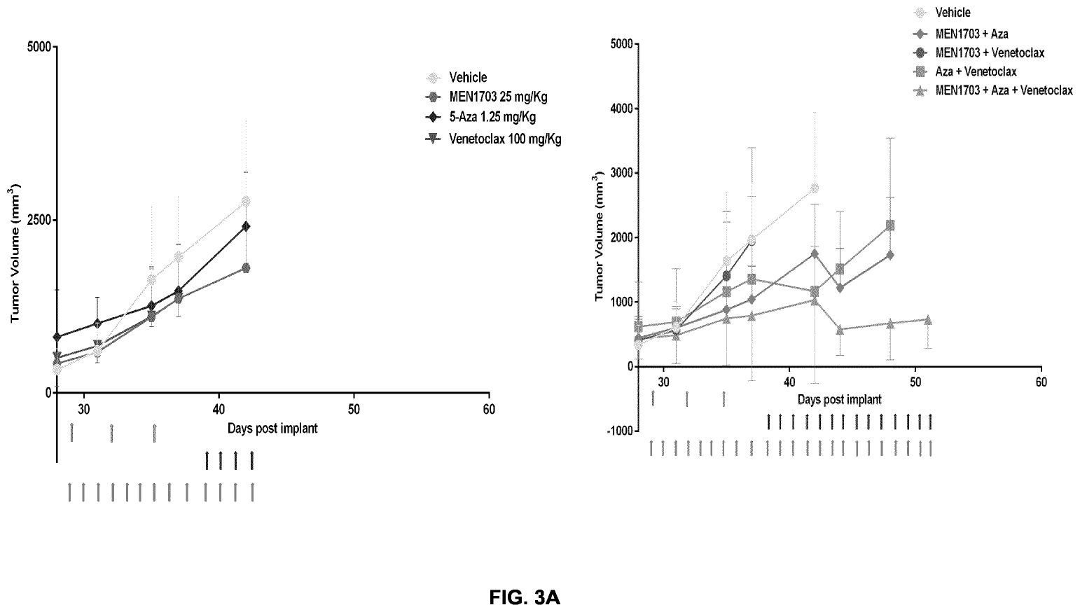

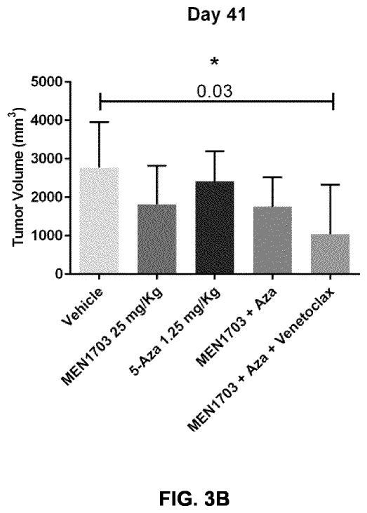

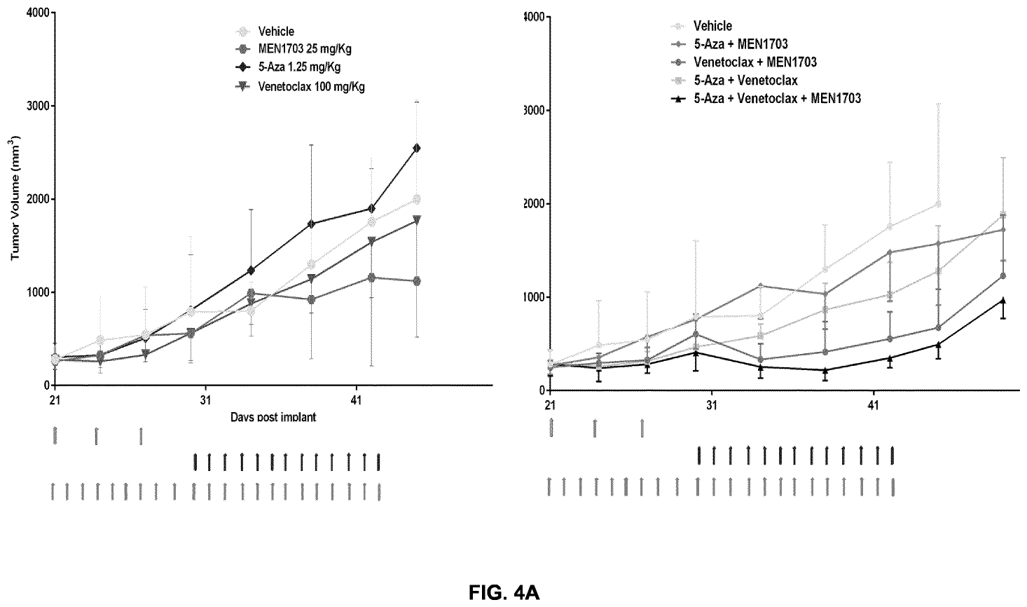

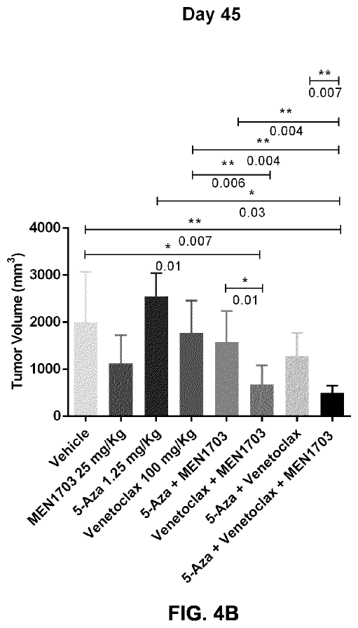

In vivo xenograft studies with MOLM-16 (FLT3 wt) and MV4-11 (FLT3 ITD) cells confirmed the in vitro results. In the MOLM-16 xenograft, consistent tumor growth inhibition was observed with the triple combination, which was the only combination statistically significant when compared to the control (see FIGS. 3A and 3B). It is noted, however, that the three actives were used at lower concentrations in this experiment (see Materials and Methods section below) because it is otherwise not possible to observe an effect, i.e. there would be no effect when combining the actives at higher concentrations. This explains why the single agents at such low concentrations were not that effective in the present experimental setup. Furthermore, high concentrations of these actives are typically toxic when administered in combination in mice. In the MV4-11 xenograft, consistent tumor growth inhibition was observed with the triple combination, which was significantly different when compared to the control, the single agent Aza and Ven treatments, and the Ven+Aza as well as the Men+Aza combination treatments. Ven+Men was in this setup also significantly different from the control and the single agent Ven treatment (see FIGS. 4A and 4B).

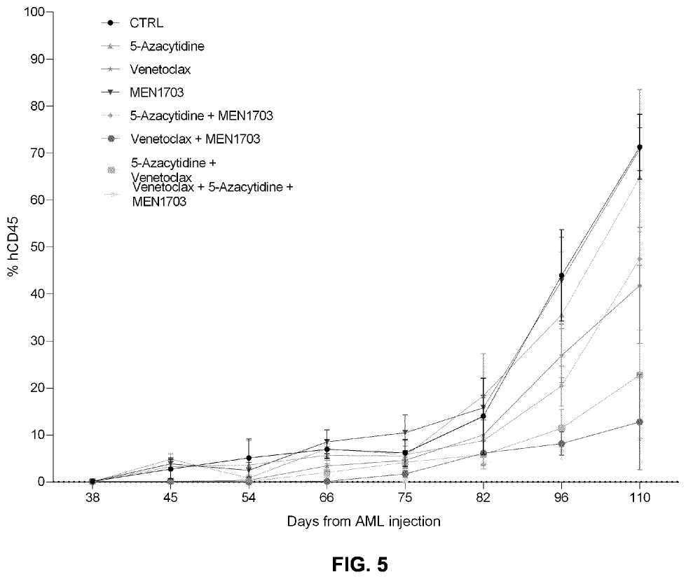

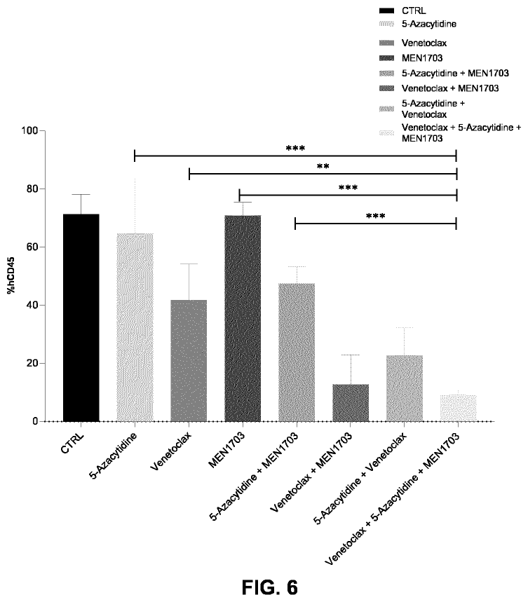

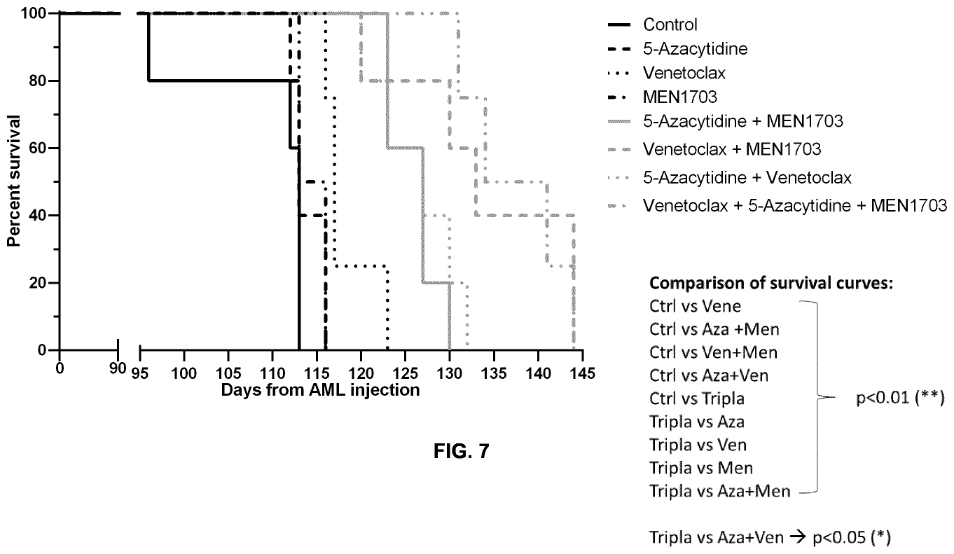

The combination treatment was also tested in an AML patient-derived xenograft (PDX) diffuse model. This model confirmed higher efficacy of the triple combination treatment vs. the Ven+Aza combination. Statistical analysis showed a significant difference in terms of tumor burden (evaluated as % of human CD45 positive cells), between the triple combination and the single agent treatments as well as the Men+Aza combination treatment (see FIGS. 5 and 6). Statistical analysis of overall survival data showed a significant difference of the triple combination treatment vs. the single agent treatments and vs. the Men+Aza or Ven+Aza combination treatments (see FIG. 7).

### Materials and Methods for Examples 1 and 2:

### Human Cell Lines

Human acute myeloid leukemia cell lines KG-1, MV4-11 and MOLM-13 were obtained from DSMZ, Braunschweig, Germany (ACC14, ACC102 and ACC554). KG-1 and MV4-11 cells were cultured in RPMI 1640 medium (Gibco, Life Technologies, Carlsbad, CA, USA) supplemented with 10% fetal bovine serum (FBS) (Sigma, Saint Louis, CA, USA). MOLM-13 cells were grown in RPMI with 20% FBS. All cells were incubated at 37° C., 5% CO2, 80% relative humidity. The human acute myeloid leukemia cell line MOLM-16 was obtained from DSMZ, Braunschweig, Germany (cell line number ACC-555. MOLM-16 cells were grown in RPMI with 20% FBS (Sigma, Saint Louis, CA, USA). Cells were incubated at 37° C., 5% CO2, 80% relative humidity.

### Actives in the Cytotoxicity Assays

Men (alternatively referred to as “MEN1703”, in the HCI-salt form (CAS number 2769008-22-) with the IUPAC name 5,6-dibromo-4-nitro-2-(piperidin-4-yl)-1-(propan-2-yl)-1H-1,3-benzodiazole-4-amine hydrochloride (batch no. A/2201/24/1)), synthesized at Aptuit (VR) and Venetoclax (alternatively referred to as “Ven” or “ABT-199”, ChemCruz no. SC-472284A) were dissolved in DMSO, divided into aliquots and stored at-80° C. until use. Stock solutions were added at appropriate concentration in culture medium before the addition to cells. 5-Azacitidine (alternatively referred to as “5-Aza” or “A2385”, Sigma Aldrich, St. Louis, MO USA), was dissolved in distilled pure water and used immediately after resuspension (since it is very unstable in aqueous solutions). A stock solution was added at appropriate concentration in culture medium before the addition to cells.

### Actives in the Mice Models

Men (alternatively referred to as “MEN1703”, in the HCI-salt form (CAS number 2769008-22-) with the IUPAC name 5,6-dibromo-4-nitro-2-(piperidin-4-yl)-1-(propan-2-yl)-1H-1,3-benzodiazole-4-amine hydrochloride (batch 76608X, synthesized at MENARINI RICERCHE SpA Pisa) was dissolved in sterile water. 5-Azacytidine (batch BCCC8387) was purchased from SIGMA Aldrich and the powder was dissolved in sterile saline solution and administered within one hour. Venetoclax (batch B0521) was purchased from Santa Cruz Biotechnology, Inc. and the powder was first dissolved in DMSO and then diluted in a solution of 20% 2-hydroxypropyl-β-cyclodextrin (HPCD) in 100 mM citrate buffer (pH 3).

### Cytotoxicity Assay

For cytotoxicity studies cells were seeded at appropriate density (50.000 cells/well) prior to addition of Ven and 5-Aza at day 0. 5-Aza was added every 24 hours due to degradation (Hollen-bach PW, 2010 and Chang E, 2016). Then, after 48 hours, Men was added to cultured cells for the final 24 h (overall, a period of 72 h was tested). The concentration range was previously identified in single agent cytotoxic assay for the same time points in order to include in the combination experiment the IC10, IC25, IC50 and IC75 values for each drug and for each AML cell lines (see Tables 1 to 3). All possible combinations were assessed: Men+Ven, Ven+5-Aza, Men+5-Aza and men+5-Aza+Ven (mav). After 72 hours, CellTiter 96 Aqueous One Solution Reagent (MTS) (Promega, Madison. WI, USA) was added to assess the cell viability. Fluorescence was measured 4h later using Tecan Infinite M200 (Tecan Trading AG, Switzerland), recording absorbance at 490 nm. Quantitative measurement of synergism/antagonism was evaluated with Combination Index (CI) on the Fraction affected (Fa) using CompuSyn (ComboSyn, Inc. Paramus, NJ, USA) (Chou TC, 2006).

### MOLM-16 Model

For the AML xenograft model, 10×10⁶ MOLM-16 cells were re-suspended in 0.2 ml of BME type III (Trevigen) at 5.6 mg/ml+DPBS (1:1) and then injected subcutaneously into the right flank of 6-8 weeks old female SCID mice (Charles River, Calco, Italy).

After injection, mice were maintained in micro isolator cages under continuously monitored environmental conditions. Drinking water and specific sterilized diet (VRF1, Charles River) were supplied ad libitum. Environmental conditions, as well as the procedures for housing and handling the animals, complied with the UKCCCR guideline (Workman P et al., 2010) and the European Convention for the protection of vertebrate animals used for experimental and other scientific purposes (Directive 2010/63/EU; 2010). Twice a week, tumor growth and body weight were evaluated and recorded.

The outcome evaluation was carried out as follows: tumor volumes were measured by caliper, and tumor masses were calculated using the following formula: [length (mm)×width² (mm)×d]/2, assuming density, d=1 mg/mm³ for tumor tissue (Teicher B. Totowa, 1997). When the average tumor volume reached 200-300 mm³ (corresponding to day 29 in FIG. 3A) animals were randomly assigned into eight groups (5-6 mice/group) and the following treatments were administered starting from day 29: Group I received vehicle (Venetoclax diluent solution) per os once daily until day 42 (single administrations, see left graph of FIG. 3A; combination administrations, see right graph of FIG. 3A), group II received Men at 25 mg/kg per os once daily q1dx5 starting from day 38 until day 42 (single administrations, see left graph of FIG. 3A), group III received 5-Azacytidine at 1.25 mg/kg intraperitoneally, given every two days for 3 total doses (single administrations, see left graph of FIG. 3A; combination administrations, see right graph of FIG. 3A), and group IV received Venetoclax at 100 mg/kg given per os once daily q1dx14 until day 42 (single administrations, see left graph of FIG. 3A), group V received the combination of Men at 25 mg/kg per os once daily q1dx14 starting from day 38 until day 51 +5-Aza at the same dose and regimen as the single agent group (combination administrations, see right graph of FIG. 3A), group VI received the combination of Men at 25 mg/kg per os once daily q1dx14 starting from day 38 until day 51+Venetoclax at 100 mg/kg given per os once daily q1dx23 starting from day 29 until day 51 (combination administrations, see right graph of FIG. 3A), group VII received the combination of 5-Aza at the same dose and schedule as the single agent group+Venetoclax at 100 mg/kg given per os once daily q1dx23 starting from day 29 until day 51 (combination administrations, see right graph of FIG. 3A, and group VIII received the triple combination of Men+5-Aza+Ven, all drugs administered at the same dose and schedule of the double combination groups.

The treatment efficacy was assessed as TVI % in treated versus control mice, using the following formula: TVI %=(1−Volume Average of treated tumor mass/Volume Average of control tumor)*100.

Mice were sacrificed when tumors reached a volume of around 10% of total body weight or when mice's body weight decreased by more than 20% compared to control animals for 7 days or more. Animals were euthanized with carbon dioxide exposure according to the standard procedures (Annex IV of Directive 2010/63/EU; 2010).

### MV4-11 Model

For the AML xenograft model, 10×10⁶ MV4-11 cells were re-suspended in 0.2 ml of BME type III (Trevigen) at 5.6 mg/ml+DPBS (1:1) and then injected subcutaneously into the right flank of 6-8 weeks old female SCID mice (Charles River, Calco, Italy).

After injection, mice were maintained in micro isolator cages under continuously monitored environmental conditions. Drinking water and specific sterilized diet (VRF1, Charles River) were supplied ad libitum. Environmental conditions, as well as the procedures for housing and handling the animals, complied with the UKCCCR guideline (Workman P et al., 2010) and the European Convention for the protection of vertebrate animals used for experimental and other scientific purposes (Directive 2010/63/EU; 2010). Twice a week, tumor growth and body weight were evaluated and recorded.

The outcome evaluation was carried out as follows: tumor volumes were measured by caliper, and tumor masses were calculated using the following formula: [length (mm)×width² (mm)×d]/2, assuming density, d=1 mg/mm³ for tumor tissue (Teicher B. Totowa, 1997). When the average tumor volume reached 200-300 mm³ (corresponding to day 21 in FIG. 4A) animals were randomly assigned into eight groups (5-6 mice/group) and the following treatments were administered starting from day 21: Group I received vehicle (Venetoclax diluent solution) per os once daily until day 42, group II received Men at 25 mg/kg per os once daily q1dx13 starting from day 30 until day 42, (single administrations, see left graph of FIG. 4A; combination administrations, see right graph of FIG. 4A), group III received 5-Azacytidine at 1.25 mg/kg intraperitoneally given every two days for 3 total doses (single administration, see left graph of FIG. 4A; combination administration see right graph of FIG. 4A), and group IV received Venetoclax at 100 mg/kg given per os once daily q1dx22 until day 42 (single administration, see left graph of FIG. 4A; combination administration, see right graph of FIG. 4A), groups V-VI-VII received the combinations of Men+5-Aza, Men+Ven, and 5-Aza+Ven, respectively, and group VIII received the triple combination of Men+5-Aza+Ven, wherein the afore-mentioned dosages and regimen of the active agents were used in the combinations as well.

The treatment efficacy was assessed as TVI % in treated versus control mice, using the following formula: TVI %=(1−Volume Average of treated tumor mass/Volume Average of control tumor)*100.

Mice were sacrificed when tumors reached a volume of around 10% of total body weight or when mice's body weight decreased by more than 20% compared to control animals for 7 days or more. Animals were euthanized with carbon dioxide exposure according to the standard procedures (Annex IV of Directive 2010/63/EU; 2010).

### AML PDX Model

The human AML sample used in this study has been tested and the results indicate a mutational status for IDH2, JAK2, MIR636, SRSF2 and RUNX1. Peripheral blood mononuclear cell (PBMC) from patient sample was isolated by Ficoll separation and subjected to T cell depletion using anti-CD3 magnetic beads (Miltenyi Biotec). T cell depletion has previously been shown to enhance the engraftment of AML cells in female non-obese diabetic severe combined immunodeficient NOD/SCID interleukin-2 receptor γ(IL-2Rγ)-null (NSG) hosts by limiting polyclonal donor T cell expansion due to xenogeneic graft versus host disease (Von Bonin M. et al., 2013). For the PDXs, experiments were carried out on 6 to 8-weeks-old female NSG mice. Mice were housed under pathogen-free conditions in the animal facilities at the European Institute of Oncology-Italian Foundation for Cancer Research Institute of Molecular Oncology (IEO-IFOM, Milan, Italy). All animal experiments were carried out in strict accordance with the Italian laws (D.L.vo 26/2014 and following additions) and approved by the institutional committee.

10 million T cell-depleted cells were transplanted intraperitoneally in 6-to 8-week-old males and females NSG mice conditioned with 1 Gy of irradiation. Bone marrow and spleen cells were collected from one primary recipient mouse and cryopreserved in liquid nitrogen after checking human engraftment (hCD45 >80%) and patient markers by FACS analysis following staining with different anti-human CD45 APC, CD33 APC-Cy7, CD13 PE-Cy7, CD117 PE and anti-mouse CD45 FITC antibodies. For the experiments, human blasts collected from spleen of second passage of xenotransplantation were injected intravenously (750.000 cells/mouse) into the lateral tail vein of NSG mice 1 day before they received low-dose irradiation (1 Gy). At day 38 post-graft, once a systemic xenograft was confirmed (around 0.05%), mice were randomized to receive treatments. The percentage of human cells in peripheral blood from tail vein was assessed by flow cytometry, once a week until day 110, evaluating anti-human CD45-APC (clone J.33, Beckman-Coulter) and anti-mouse CD45-PE (clone 30-F11, BD) to exclude murine cells contamination. All blood samples were lysed with ammonium chloride red-blood-cell buffer (Qiagen) prior to staining. Percentages of stained cells were determined and compared to appropriate negative controls. Seven-aminoactinomycin D (7AAD) from Sigma-Aldrich was used to enumerate viable, apoptotic, and dead cells. Cell suspensions was evaluated by a 3-laser, 10-colour flow cytometer (Navios, Beckman Coulter, Brea, CA, USA) using analysis gates designed to exclude dead cells, platelets, and debris. Engrafted mice (n=5 per study arm) were treated with either vehicle or with different drugs used as single agents or in combination.

In detail, mice were treated with 5-azacytidine (1.25 mg/kg) intraperitoneally (i.p.) for 3 days (on day 1-4-7 post engraftment) or MEN1703 (25 mg/kg) for 14 consecutive days (day 10-23 post engraftment) or Venetoclax (100 mg/kg) for 23 consecutive days (starting on day 1 post-engraftment), both via oral gavage.

Treatment effectiveness was assessed as frequency of human CD45⁺ cells in peripheral blood of mice. Animals were euthanized with carbon dioxide exposure according to the standard procedure.

### Statistical Analyses

GraphPad Prism software (GraphPAD Software Inc., California) was used for statistical analysis. Statistical differences were considered to be significant at p-value <0.05 using the two-tailed Mann-Whitney rank test. In vivo data (FIGS. 4 to 6) are presented as mean, with value for each group represented as a symbol of different shapes and lines of different colors. Analysis at day 110 of AML PDX model, Tukey's multiple comparison test was used.

### Example 3: In Vitro Studies in Cell Lines for a Combination with S65487

In an in vitro standard cytotoxicity experiment, two AML cell lines (MOLM-13 [FLT3 ITD] and MV4-11 [FLT3 ITD]) were treated for 72 hours with MEN and S65487 either as a single agent or in combination. The synergistic effect of the combination was measured according to the Combination Index calculation (Chou TC, 2006).

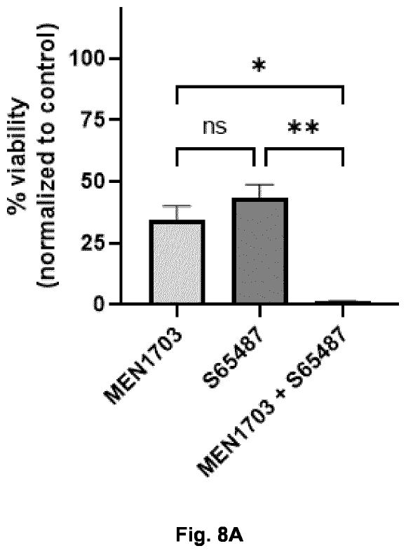

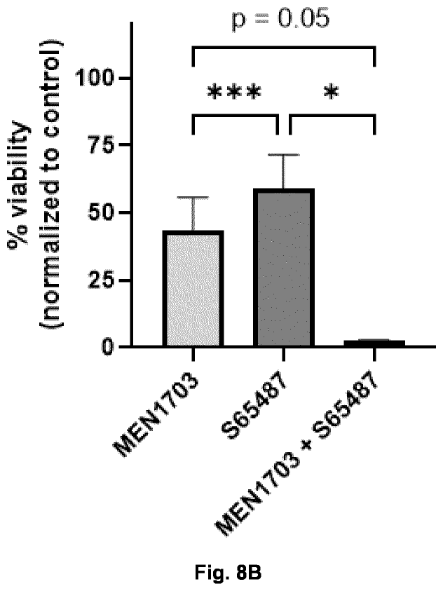

The synergistic effect of the combination of MEN and S65487 in MOLM13 cells is shown in Table 4 (see Table 4, Combination Index or CI<1.0). The combination of MEN and S65487 at the IC50 concentrations induced a cytotoxicity significantly different from S65487-induced cytotoxicity in MOLM-13 cells and the MEN 1703-induced cytotoxicity in MOLM-13 cells (Tukey's multiple comparison one-way ANOVA test; p<0.05*, p<0.01**, see FIG. 8A).

The combination of MEN and S65487 was synergistic in MV4-11 cells, as can be derived from Table 5 (see Table 5, Combination Index or CI<1.0). The combination of MEN and S65487 at the IC50 concentrations induced a cytotoxicity significantly different from S65487-induced cytotoxicity in MV4-11 cells and the MEN 1703-induced cytotoxicity in MV4-11 cells (Tukey's multiple comparison one-way ANOVA test; p<0.05*, p<0.001***, see FIG. 8B).

TABLE 4
in vitro Combination Index in MOLM-13 cells

| | | Combination Index | | | |
|---|---|---|---|---|---|
| | | MEN IC10 | MEN IC25 | MEN IC50 | MEN IC75 |
| | | 0.08729 μM | 0.2111 μM | 0.5104 μM | 1.234 μM |
| S65487 IC10 | 0.0145 μM | 1.14 | 0.46 | 0.25 | 0.16 |
| S65487 IC25 | 0.0570 μM | 0.27 | 0.15 | 0.07 | 0.13 |
| S65487 IC50 | 0.2245 μM | 0.09 | 0.05 | 0.06 | 0.13 |
| S65487 IC75 | 0.8800 μM | 0.05 | 0.03 | 0.06 | 0.14 |

TABLE 5
in vitro Combination Index in MV4-11 cells

| | | Combination Index | | | |
|---|---|---|---|---|---|
| | | MEN IC10 | MEN IC25 | MEN IC50 | MEN IC75 |
| | | 0.01315 μM | 0.0350 μM | 0.0930 μM | 0.2486 μM |
| S65487 IC10 | 0.0025 μM | 0.66 | 0.70 | 0.67 | 0.46 |
| S65487 IC25 | 0.0066 μM | 0.53 | 0.51 | 0.41 | 0.38 |
| S65487 IC50 | 0.0180 μM | 0.67 | 0.56 | 0.33 | 0.42 |
| S65487 IC75 | 0.0480 μM | 1.06 | 0.74 | 0.47 | 0.60 |

### Example 4: In Vitro Studies in Cell Lines for a Combination with AZD4320

In an in vitro standard cytotoxicity experiment, two AML cell lines (MOLM-13 [FLT3 ITD] and MV4-11 [FLT3 ITD]) were treated for 72 hours with MEN and AZD4320 either as a single agent or in combination. The synergistic effect of the combination was measured according to the Combination Index calculation (Chou TC, 2006).

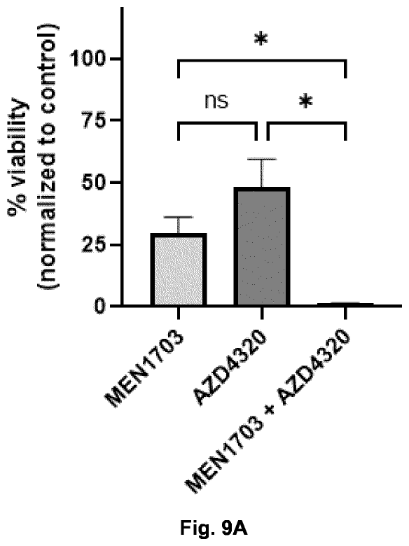

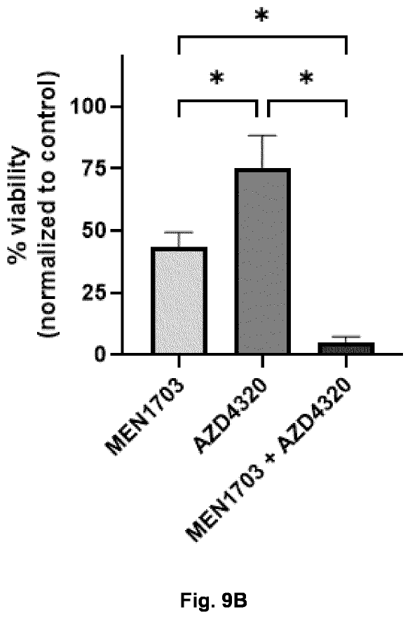

The synergistic effect of the combination of MEN and S65487 in MOLM13 cells is shown in Table 6 (see Table 6, Combination Index or CI<1.0). The combination of MEN and AZD4320 at the IC50 concentrations induced a cytotoxicity significantly different from AZD4320-induced cytotoxicity in MOLM-13 cells and the MEN 1703-induced cytotoxicity in MOLM-13 cells (Tukey's multiple comparison one-way ANOVA test; p<0.05*, see FIG. 9A).

The combination of MEN and AZD4320 was synergistic in MV4-11 cells, as can be derived from Table 7 (see Table 7, Combination Index or CI<1.0). The combination of MEN and AZD4320 at the IC50 concentrations induced a cytotoxicity significantly different from AZD4320-induced cytotoxicity in MV4-11 cells and the MEN 1703-induced cytotoxicity in MV4-11 cells (Tukey's multiple comparison one-way ANOVA test; p<0.05*, see FIG. 9B).

TABLE 6
in vitro Combination Index in MOLM-13 cells

| | | Combination Index | | | |
|---|---|---|---|---|---|
| | | MEN IC10 | MEN IC25 | MEN IC50 | MEN IC75 |
| | | 0.08729 μM | 0.2111 μM | 0.5104 μM | 1.234 μM |
| AZD4320 IC10 | 0.0034 μM | 1.72 | 1.16 | 0.61 | 0.23 |
| AZD4320 IC25 | 0.0110 μM | 1.22 | 0.45 | 0.16 | 0.14 |
| AZD4320 IC50 | 0.0370 μM | 0.37 | 0.10 | 0.06 | 0.11 |
| AZD4320 IC75 | 0.1200 μM | 0.08 | 0.03 | 0.05 | 0.12 |

TABLE 7
in vitro Combination Index in MV4-11 cells

| | | Combination Index | | | |
|---|---|---|---|---|---|
| | | MEN IC10 | MEN IC25 | MEN IC50 | MEN IC75 |
| | | 0.01315 μM | 0.0350 μM | 0.0930 μM | 0.2486 μM |
| AZD4320 IC10 | 0.00065 μM | 1.01 | 0.71 | 0.64 | 0.45 |
| AZD4320 IC25 | 0.0013 μM | 0.60 | 0.63 | 0.52 | 0.36 |
| AZD4320 IC50 | 0.0027 μM | 0.73 | 0.65 | 0.39 | 0.37 |
| AZD4320 IC75 | 0.0055 μM | 0.83 | 0.64 | 0.35 | 0.43 |

### Materials and Methods for Examples 3 and 4

### Human Cell Lines

Human acute myeloid leukemia cell lines MV4-11 and MOLM-13 were obtained from DSMZ, Braunschweig, Germany (ACC102 and ACC554). MV4-11 cells were cultured in RPMI 1640 medium (Gibco, Life Technologies, Carlsbad, CA, USA) supplemented with 10% fetal bovine serum (FBS) (Sigma, Saint Louis, CA, USA). MOLM-13 cells were grown in RPMI with 20% FBS. All cells were incubated at 37° C., 5% CO₂, 80% relative humidity.

### Drugs

MEN1703 (alternatively referred to as “Men”, “MEN”, “MEN1703” or “SEL24/MEN1703”), in the HCl-salt form (CAS number 2769008-22-) with the IUPAC name 5,6-dibromo-4-nitro-2-(piperidin-4-yl)-1-(propan-2-yl)-1H-1,3-benzodiazole-4-amine hydrochloride (batch no. A/2201/24/1)), synthesized at Aptuit (VR); AZD4320 from MedChem Express, Monmouth Junction, NJ, USA, batch no. 64354) and S65487 from MedChem Express, Monmouth Junction, NJ, USA were dissolved in DMSO, divided into aliquots and stored at −80° C. until use. Stock solutions were added at appropriate concentration in culture medium before the addition to cells.

### Cytotoxicity

For cytotoxicity studies cells were seeded at appropriate density (50,000 cells/well) prior to addition of MEN and Quiz at day 0. The concentration range was previously identified in single agent cytotoxicity assays for the same time points in order to include in the combination experiment the IC10, IC25, IC50 and IC75 values for each drug and for each AML cell lines (see Tables 4 to 7). After 72 hours, CellTiter 96 Aqueous One Solution Reagent (MTS) (Promega, Madison. WI, USA) was added to assess the cell viability. Fluorescence was measured 4 h later using Tecan Infinite M200 (Tecan Trading AG, Switzerland), recording absorbance at 490 nm. Quantitative measurement of synergism/antagonism was evaluated with Combination Index (CI) on the Fraction affected (Fa) using CompuSyn (ComboSyn, Inc. Paramus, NJ, USA) (Chou TC, 2006).

### 4. REFERENCES

Arulananda et al., A novel BH3-mimetic, AZD0466, targeting BCL-XL and BCL-2 is effective in pre-clinical models of malignant pleural mesothelioma, Cell Death Discovery, 2021,7, 122-131.
Balachander et a., AZD4320, a dual inhibitor of Bcl-2 and Bcl-XL, induces tumor regression in hematological cancer models without dose-limiting thrombocytopenia, Clin Cancer Res, 2020, 26(24): 6535-6549
Chang E. et al., The combination of FLT3 and DNA methyltransferase inhibition is synergistically cytotoxic to FLT3/ITD acute myeloid leukemia cells, Leukemia, 2016
Chou TC, Theoretical basis, experimental design, and computerized simulation of synergism and antagonism in drug combination studies, Pharmacol Rev, 2006
Directive 2010/63/Eu of The European Parliament And Of The Council On The Protection Of Animals Used For Scientific Purposes, 22 Sep. 2010
Hollenbach PW, et al., A comparison of azacitidine and decitabine activities in acute myeloid leukemia cell lines, PLoS One, 2010
Kipp D. and Wei A., The path to approval for oral hypomethylating agents in acute myeloid leukemia and myelodysplastic syndromes, Future Oncol. 2021, 17(20), 2563-2571
Nakao M, et al., Internal tandem duplication of the flt3 gene found in acute myeloid leukemia. Leukemia, 1996, 10 (12), 1911-1918
Saliba A. et al., Resistance to Venetoclax and hypomethylating agents in acute myeloid leukemia, Cancer Drug Resistance, 2021, 4, 125-42
Samra B, et al., Venetoclax-based combinations in Acute Myeloid Leukemia: Current Evidence and Future Directions, Frontiers in Oncology, 2020, 10, Article 562558
Teicher B. Totowa. Anticancer Drug Development Guide. New Jersey: Humana Press, 1997
Workman P., et al., Guidelines for The Welfare And Use Of Animals In Cancer Research. British Journal Of Cancer, 2010, 102, 1555-1577
Zhang et al., Rapid and efficient response to gilteritinib and Venetoclax-based therapy in two AML patients with FLT3-ITD mutation unresponsive to Venetoclax plus azacitidine, Onco Targets and Therapy, 2022:15, 159-164
Von Bonin M, Wermke M, Cosgun KN, et al. In vivo expansion of co-transplanted T cells impacts on tumor re-initiating activity of human acute myeloid leukemia in NSG mice. PLoS One. 10 2013;8(4):e60680

1. A combination of (i) a BCL-2 inhibitor and (ii) SEL24/MEN1703 for use as medicament.

2. A combination of (i) a BCL-2 inhibitor and (ii) SEL24/MEN1703 for use in the treatment of a patient suffering from cancer.

3. The combination for use according to claim 2, wherein the cancer is a hematological cancer.

4. The combination for use according to claim 2 or 3, wherein the cancer is acute myeloid leukemia (AML).

5. The combination for use according to any one of the preceding claims, wherein the BCL-2 inhibitor is Venetoclax.

6. The combination for use according to claim 5, wherein Venetoclax is administered at a daily dose of about 50 mg to about 600 mg.

7. The combination for use according to any one of the preceding claims, wherein SEL24/MEN1703 is administered at a daily dose of about 50 mg to about 150 mg.

8. The combination for use according to any one of the preceding claims, wherein the combination further comprises (iii) a hypomethylating agent or cytarabine.

9. The combination for use according to claim 8, wherein the hypomethylating agent is selected from the group consisting of azacitidine, decitabine, CC-486 and ASTX727.

10. The combination for use according to claim 8 or 9, wherein the hypomethylating agent is administered at a daily dose of about 10 mg/m² to about 100 mg/m².

11. The combination for use according to claim 10, where the hypomethylating agent is azacitidine and the azacitidine is administered at a daily dose of about 75 mg/m².

12. The combination for use according to claim 10, where the hypomethylating agent is decitabine and the decitabine is administered at a daily dose of about 20 mg/m².

13. The combination for use according to claim 8, wherein cytarabine is administered at a daily dose of about 20 mg/m².

14. A kit of dosage forms comprising (i) a dosage form comprising a BCL-2 inhibitor and (ii) a dosage form comprising SEL24/MEN1703.

15. A dosage form comprising (i) a BCL-2 inhibitor and (ii) SEL24/MEN1703.
